(19) United States
(12) Patent Application Publication
(10) Pub. No.: US 2008/0058793 A1
(43) Pub. Date: Mar. 6, 2008

(54) Electromagnetic apparatus for prophylaxis and repair of ophthalmic tissue and method for using same

(76) Inventors: Arthur A. Pilla, Oakland, NJ (US); Andre Dimino, Woodcliff Lake, NJ (US)

Correspondence Address: LEN TAYLOR, PATENT ATTORNEY, 261 DAVENPORT STREET, SOMERVILLE, NJ, 08876, US

(21) Appl. No.: 11/818,065

(22) Filed: Jun. 12, 2007

Related U.S. Application Data
(60) Provisional application No. 60/812,841, filed on Jun. 12, 2006.

(51) Int. Cl. A61B 18/18

(52) U.S. Cl. 606/33

(57) ABSTRACT

An apparatus and method for altering the electromagnetic environment of ophthalmic tissues, cells, and molecules comprising, establishing baseline thermal fluctuations in voltage and electrical impedance at a target pathway structure depending on a state of at least one of the ophthalmic tissue components, configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance, generating an electromagnetic signal from the configured at least one waveform, and coupling the electromagnetic signal to the target pathway structure using a coupling device whereby enhancing the release of second messengers, such as NO, growth factors and cytokines at the target pathway structure. The use of the within specified electromagnetic waveforms can have particular utility in the prophylactic treatment of ophthalmic tissue and the treatment of ophthalmic diseases such as macular degeneration, glaucoma, retinosa pigmentosa, repair and regeneration of optic nerve prophylaxis and other related diseases that respond positively to the physiological effects of these waveforms.

This application claims the benefit of U.S. Provisional Application 60/812,841 filed Jun. 12, 2006.

### BACKGROUND OF THE INVENTION

### Field of the Invention

This invention relates to delivering electromagnetic signals to ophthalmic tissue of humans and animals that are injured or diseased whereby the interaction with the electromagnetic environment of living tissues, cells, and molecules is altered to achieve a therapeutic or wellness effect. The invention also relates to a method of modification of cellular and tissue growth, repair, maintenance and general behavior by the application of encoded electromagnetic information. More particularly, this invention provides for an application of highly specific electromagnetic frequency (“EMF”) signal patterns to ophthalmic tissue by surgically non-invasive reactive coupling of encoded electromagnetic information. Such application of electromagnetic waveforms to human and animal target pathway structures such as cells, organs, tissues and molecules, can serve to remedy injured or diseased ophthalmic tissue or to prophylactically treat such tissue.

The use of most low frequency EMF has been in conjunction with applications of bone repair and healing. As such, EMF waveforms and current orthopedic clinical use of EMF waveforms comprise relatively low frequency components inducing maximum electrical fields in a millivolts per centimeter (mV/cm) range at frequencies under five KHz. A linear physicochemical approach employing an electrochemical model of cell membranes to predict a range of EMF waveform patterns for which bioeffects might be expected is based upon an assumption that cell membranes, and specifically ion binding at structures in or on cell membranes or surfaces, are a likely EMF target. Therefore, it is necessary to determine a range of waveform parameters for which an induced electric field could couple electrochemically at a cellular surface, such as by employing voltage-dependent kinetics.

A pulsed radio frequency (“PRF”) signal derived from a 27.12 MHz continuous sine wave used for deep tissue healing is known in the prior art of diathermy. A pulsed successor of the diathermy signal was originally reported as an electromagnetic field capable of eliciting a non-thermal biological effect in the treatment of infections. Subsequently, PRF therapeutic applications have been reported for the reduction of post-traumatic and post-operative pain and edema in soft tissues, wound healing, burn treatment, and nerve regeneration. The application of PRF for resolution of traumatic and chronic edema has become increasingly used in recent years. Results to date using PRF in animal and clinical studies suggest that edema may be measurably reduced from such electromagnetic stimulus.

The within invention is based upon biophysical and animal studies that attribute effectiveness of cell-to-cell communication on tissue structures' sensitivity to induced voltages and associated currents. A mathematical analysis using at least one of a Signal to Noise Ratio (“SNR”) and a Power Signal to Noise Ratio (“Power SNR”) evaluates whether EMF signals applied to target pathway structures such as cells, tissues, organs, and molecules, are detectable above thermal noise present at an ion binding location. Prior art of EMF dosimetry did not take into account dielectric properties of tissue structures, rather the prior art utilized properties of isolated cells. By utilizing dielectric properties, reactive coupling of electromagnetic waveforms configured by optimizing SNR and Power SNR mathematical values evaluated at a target pathway structure can enhance wellness of the ophthalmic system as well as repair of various ophthalmic injuries and diseases in human and animal cells, organs, tissues and molecules for example wet macular degeneration and dry macular degeneration. Cell, organ, tissue, and molecule repair enhancement results from increased blood flow and anti-inflammatory effects, and modulation of angiogenesis and neovascularization as well as from other enhanced bioeffective processes such as growth factor and cytokine release.

Recent clinical use of non-invasive PRF at radio frequencies has used pulsed bursts of a 27.12 MHz sinusoidal wave, each pulse burst typically exhibiting a width of sixty five microseconds and having approximately 1,700 sinusoidal cycles per burst, and with various burst repetition rates.

Broad spectral density bursts of electromagnetic waveforms having a frequency in the range of one hertz (Hz) to one hundred megahertz (MHz), with 1 to 100,000 pulses per burst, and with a burst-repetition rate of 0.01 to 10,000 Hertz (Hz), are selectively applied to human and animal cells, organs, tissues and molecules. The voltage-amplitude envelope of each pulse burst is a function of a random, irregular, or other like variable, effective to provide a broad spectral density within the burst envelope. The variables are defined by mathematical functions that take into account signal to thermal noise ratio and Power SNR in specific target pathway structures. The waveforms are designed to modulate living cell growth, condition and repair. Particular applications of these signals include, but are not limited to, enhancing treatment of organs, muscles, joints, eyes, skin and hair, post surgical and traumatic wound repair, angiogenesis, improved blood perfusion, vasodilation, vasoconstriction, edema reduction, enhanced neovascularization, bone repair, tendon repair, ligament repair, organ regeneration and pain relief. The application of the within electromagnetic waveforms can serve to enhance healing of various ophthalmic tissue injuries and diseases, as well as provide prophylactic treatment for such tissue.

According to an embodiment of the present invention a pulse burst envelope of higher spectral density can more efficiently couple to physiologically relevant dielectric pathways, such as cellular membrane receptors, ion binding to cellular enzymes, and general transmembrane potential changes. An embodiment according to the present invention increases the number of frequency components transmitted to relevant cellular pathways, resulting in different electromagnetic characteristics of healing tissue and a larger range of biophysical phenomena applicable to known healing mechanisms becoming accessible, including enhanced enzyme activity, second messenger, such as nitric oxide (“NO”) release, growth factor release and cytokine release. By increasing burst duration and by applying a random, or other high spectral density envelope, to a pulse burst envelope of mono-polar or bi-polar rectangular or sinusoidal pulses that induce peak electric fields between 10⁻⁶ and 10 volts per centimeter (V/cm), and that satisfy detectability requirements according to SNR or Power SNR, a more efficient and greater effect could be achieved on biological healing processes applicable to both soft and hard tissues in humans and animals resulting in an acceleration of ophthalmic injury and disease repair.

The present invention relates to known mechanisms of ophthalmic injury and disease repair and healing that involve the naturally timed release of the appropriate anti-inflammatory cascade and growth factor or cytokine release in each stage of wound repair as applied to humans and animals. Specifically, ophthalmic injury and disease repair involves an inflammatory phase, angiogenesis, cell proliferation, collagen production, and remodeling stages. There are timed releases of second messengers, such as NO, specific cytokines and growth factors in each stage. Electromagnetic fields can enhance blood flow and enhance the binding of ions, which, in turn, can accelerate each healing phase. It is the specific intent of this invention to provide an improved means to enhance the action of endogenous factors and accelerate repair and to affect wellness. An advantageous result of using the present invention is that ophthalmic injury and disease repair, and healing can be accelerated due to enhanced blood flow or enhanced biochemical activity. In particular, an embodiment according to the present invention pertains to using an induction means such as a coil to deliver pulsing electromagnetic fields (“PEMF”) for the maintenance of the ophthalmic system and the treatment of ophthalmic diseases such as macular degeneration, glaucoma, retinosa pigmentosa, repair and regeneration of optic nerve prophylaxis, and other related diseases. More particularly, this invention provides for the application, by surgically non-invasive reactive coupling, of highly specific electromagnetic signal patterns to one or more body parts. Such applications made on a non-invasive basis to the constituent tissues of the ophthalmic system and its surrounding tissues can serve to improve the physiological parameters of ophthalmic diseases.

It is an object of the present invention to provide an improved means to accelerate the intended effects or improve efficacy as well as other effects of the second messengers, cytokines and growth factors relevant to each stage of ophthalmic injury and disease repair and healing.

Another object of the present invention is to cause and accelerate healing for treatment of ophthalmic diseases such as wet macular degeneration, dry macular degeneration, glaucoma, retinosa pigmentosa, repair and regeneration of optic nerve, prophylaxis, and other related diseases.

Another object of the present invention is to accelerate healing of ophthalmic injuries of any type.

Another object of the present invention is to maintain wellness of the ophthalmic system.

Another object of the present invention is that by applying a high spectral density voltage envelope as a modulating or pulse-burst defining parameter according to SNR and Power SNR requirements, power requirements for such increased duration pulse bursts can be significantly lower than that of shorter pulse bursts having pulses within the same frequency range; this results from more efficient matching of frequency components to a relevant cellular/molecular process. Accordingly, the advantages of enhanced transmitted dosimetry to relevant dielectric pathways and of decreased power requirements, are achieved.

Therefore, a need exists for an apparatus and a method that effectively enhances wellness of the ophthalmic system and accelerates healing of ophthalmic injuries, ophthalmic diseases, areas around the ophthalmic system by modulating ion binding at cells, organs, tissues and molecules of humans and animals.

### SUMMARY OF THE INVENTION

An embodiment according to the present invention comprises an electromagnetic signal having a pulse burst envelope of spectral density to efficiently couple to physiologically relevant dielectric pathways, such as cellular membrane receptors, ion binding to cellular enzymes, and general transmembrane potential changes. The use of a burst duration which is generally below 100 microseconds for each PRF burst, limits the frequency components that could couple to the relevant dielectric pathways in cells and tissue. An embodiment according to the present invention increases the number of frequency components transmitted to relevant cellular pathways whereby access to a larger range of biophysical phenomena applicable to known healing mechanisms, including enhanced second messenger release, enzyme activity and growth factor and cytokine release can be achieved. By increasing burst duration and applying a random, or other envelope, to the pulse burst envelope of mono-polar or bi-polar rectangular or sinusoidal pulses which induce peak electric fields between 10⁻⁶ and 10 V/cm, a more efficient and greater effect can be achieved on biological healing processes applicable to both soft and hard tissues in humans, animals and plants.

Another embodiment according to the present invention comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present invention enhance the release of such compounds thus advantageously providing an improved means to enhance prophylactic protection and wellness of living organisms. In certain ophthalmic diseases there are physiological deficiencies and disease states that can have a lasting and deleterious effect on the proper functioning of the ophthalmic system. Those physiological deficiencies and disease states can be positively affected on a non-invasive basis by the therapeutic application of waveforms configured according to an embodiment of the present invention. In addition, electromagnetic waveforms configured according to an embodiment of the present invention can have a prophylactic effect on the ophthalmic system whereby a disease condition can be prevented, and if a disease condition already exists in its earliest stages, that condition can be prevented from developing into a more advanced state.

An example of an ophthalmic disease that can be positively affected by an embodiment according to the present invention, both on a chronic disease as well on a prophylactic basis, is macular degeneration. Age-related macular degeneration (“ARMD”) is the most common cause of irreversible vision loss those over the age of 60. Macular degeneration is a disorder of the retina, the light-sensitive inner lining of the back of the eye. There are a number of abnormalities associated with the term “age-related macular degeneration.” They range from mild changes with no decrease in vision to abnormalities severe enough to result in the loss of all “straight ahead” vision. Macular degeneration does not cause total blindness because the remaining and undamaged parts of the retina around the macula continue to provide “side” vision.

There are two main types of macular degeneration, “dry” and “wet.” With respect to dry macular degeneration, aging causes the cells in the retina to become less efficient. Deposits of tissue, called drusen, appear under the retina which can be identified through visual examination. A few small drusen may cause no decrease in vision. However, if too many large drusen develop, vision will decrease. The application of electromagnetic waveforms configured according to an embodiment of the present invention can positively effect tissue present in the retina and modify the propensity to form drusen, thereby having an effect on the progression of dry macular degeneration. Conversely, wet macular degeneration is a function of leaking of the capillaries in the layer of cells below the retina called the retinal pigment epithelium. Electromagnetic waveforms configured according to an embodiment of the present invention, have proven to have a positive effect on circulatory vessels and other tissues which can lead to an improvement in the disease state of wet macular degeneration.

Another advantage of electromagnetic waveforms configured according to an embodiment of the present invention is that by applying a high spectral density voltage envelope as the modulating or pulse-burst defining parameter, the power requirement for such increased duration pulse bursts can be significantly lower than that of shorter pulse bursts containing pulses within the same frequency range; this is due to more efficient matching of the frequency components to the relevant cellular/molecular process. Accordingly, the dual advantages, of enhanced transmitted dosimetry to the relevant dielectric pathways and of decreased power requirement are achieved.

The present invention relates to a therapeutically beneficial method of and apparatus for non-invasive pulsed electromagnetic treatment for enhanced condition, repair and growth of living tissue in animals, humans and plants. This beneficial method operates to selectively change the bioelectromagnetic environment associated with the cellular and tissue environment through the use of electromagnetic means such as PRF generators and applicator heads. An embodiment of the present invention more particularly includes the provision of a flux path, to a selectable body region, of a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which the instantaneous minimum amplitude thereof is not smaller than the maximum amplitude thereof by a factor of one ten-thousandth. Further, the repetition rate of such pulse bursts may vary from 0.01 to 10,000 Hz. Additionally a mathematically-definable parameter can be employed in lieu of said random amplitude envelope of the pulse bursts.

According to an embodiment of the present invention, by treating a selectable body region with a flux path comprising a succession of EMF pulses having a minimum width characteristic of at least about 0.01 microseconds in a pulse burst envelope having between about 1 and about 100,000 pulses per burst, in which a voltage amplitude envelope of said pulse burst is defined by a randomly varying parameter in which instantaneous minimum amplitude thereof is not smaller than the maximum amplitude thereof by a factor of one ten-thousandth. The pulse burst repetition rate can vary from about 0.01 to about 10,000 Hz. A mathematically definable parameter can also be employed to define an amplitude envelope of said pulse bursts.

By increasing a range of frequency components transmitted to relevant cellular pathways, access to a large range of biophysical phenomena applicable to known healing mechanisms, including enhanced second messenger release, enzyme activity and growth factor and cytokine release, is advantageously achieved.

According to an embodiment of the present invention, by applying a random, or other high spectral density envelope, to a pulse burst envelope of mono-polar or bi-polar rectangular or sinusoidal pulses which induce peak electric fields between 10−6 and 10 volts per centimeter (V/cm), a more efficient and greater effect can be achieved on biological healing processes applicable to both soft and hard tissues in humans, animals and plants. A pulse burst envelope of higher spectral density can advantageously and efficiently couple to physiologically relevant dielectric pathways, such as, cellular membrane receptors, ion binding to cellular enzymes, and general transmembrane potential changes thereby modulating angiogenesis and neovascularization.

Another advantage of an embodiment according to the present invention is that by applying a high spectral density voltage envelope as a modulating or pulse-burst defining parameter, power requirements for such modulated pulse bursts can be significantly lower than that of an unmodulated pulse. This is due to more efficient matching of the frequency components to the relevant cellular/molecular process. Accordingly, the dual advantages of enhanced transmitting dosimetry to relevant dielectric pathways and of decreasing power requirements are achieved.

A preferred embodiment according to the present invention utilizes a Power Signal to Noise Ratio (“Power SNR”) approach to configure bioeffective waveforms and incorporates miniaturized circuitry and lightweight flexible coils. This advantageously allows a device that utilizes a Power SNR approach, miniaturized circuitry, and lightweight flexible coils, to be completely portable and if desired to be constructed as disposable and if further desired to be constructed as implantable. The lightweight flexible coils can be an integral portion of a positioning device such as surgical dressings, wound dressings, pads, seat cushions, mattress pads, wheelchairs, chairs, and any other garment and structure juxtaposed to living tissue and cells. By advantageously integrating a coil into a positioning device therapeutic treatment can be provided to living tissue and cells in an inconspicuous and convenient manner.

Specifically, broad spectral density bursts of electromagnetic waveforms, configured to achieve maximum signal power within a bandpass of a biological target, are selectively applied to target pathway structures such as living organs, tissues, cells and molecules. Waveforms are selected using a novel amplitude/power comparison with that of thermal noise in a target pathway structure. Signals comprise bursts of at least one of sinusoidal, rectangular, chaotic and random wave shapes, have frequency content in a range of about 0.01 Hz to about 100 MHz at about 1 to about 100,000 bursts per second, and have a burst repetition rate from about 0.01 to about 1000 bursts/second. Peak signal amplitude at a target pathway structure such as tissue, lies in a range of about 1 μV/cm to about 100 mV/cm. Each signal burst envelope may be a random function providing a means to accommodate different electromagnetic characteristics of healing tissue. A preferred embodiment according to the present invention comprises about 0.1 to about 100 millisecond pulse burst comprising about 1 to about 200 microsecond symmetrical or asymmetrical pulses repeating at about 0.1 to about 100 kilohertz within the burst. The burst envelope is a modified 1/f function and is applied at random repetition rates between about 0.1 and about 1000 Hz. Fixed repetition rates can also be used between about 0.1 Hz and about 1000 Hz. An induced electric field from about 0.001 mV/cm to about 100 mV/cm is generated. Another embodiment according to the present invention comprises an about 0.01 millisecond to an about 10 millisecond burst of high frequency sinusoidal waves, such as 27.12 MHz, repeating at about 1 to about 100 bursts per second. An induced electric field from about 0.001 mV/cm to about 100 mV/cm is generated. Resulting waveforms can be delivered via inductive or capacitive coupling.

It is another object of the present invention to provide an electromagnetic method of treatment of living cells and tissues comprising a broad-band, high spectral density electromagnetic field.

It is a further object of the present invention to provide an electromagnetic method of treatment of living cells and tissues comprising amplitude modulation of a pulse burst envelope of an electromagnetic signal that will induce coupling with a maximum number of relevant EMF-sensitive pathways in cells or tissues.

It is an object of the present invention to configure a power spectrum of a waveform by mathematical simulation by using signal to noise ratio (“SNR”) analysis to configure a waveform optimized to modulate angiogensis and neovascularization then coupling the configured waveform using a generating device such as ultra lightweight wire coils that are powered by a waveform configuration device such as miniaturized electronic circuitry.

It is another object of the present invention to provide multiple coils, delivering a waveform configured by SNR/Power analysis of a target pathway structure, to increase area of treatment coverage.

It is another object of the present invention to provide multiple coils that are simultaneously driven or that are sequentially driven such as multiplexed, with the same or different optimally configured waveforms as shown above.

It is a further object of the present invention to provide flexible, lightweight coils that focus the EMF signal to the affected tissue by incorporating the coils, delivering a waveform configured by SNR/Power analysis of a target pathway structure, into dressings and ergonomic support garments.

It is yet a further object of the present invention to utilize lightweight flexible coils or conductive thread to deliver the EMF signal to affected tissue by incorporating such coils or conductive threads as an integral part of various types of bandages, such as, compression, elastic, cold compress and hot compress and delivering a waveform configured by SNR/Power analysis of a target pathway structure.

It is yet a further object of the present invention to incorporate at least one coil in a surgical wound dressing to apply an enhanced EMF signal non-invasively and non-surgically, the surgical wound dressing to be used in combination with standard wound treatment.

It is another object of the present invention to construct the coils delivering a waveform configured by SNR/Power analysis of a target pathway structure, for easy attachment and detachment to dressings, garments and supports by using an attachment means such as Velcro®, an adhesive and any other such temporary attachment means.

A further object of the present invention is to integrate at least one coil delivering a waveform configured by SNR/Power analysis of a target pathway structure, with a therapeutic surface, structure or device to enhance the effectiveness of such therapeutic surface, structure or device to augment the activity of cells and tissues of any type in any living target area.

It is yet a further object of the present invention to provide an improved electromagnetic method of the beneficial treatment of living cells and tissue by the modulation of electromagnetically sensitive regulatory processes at the cell membrane and at junctional interfaces between cells.

A further object of the present invention is to provide a means for the use of electromagnetic waveforms to cause a beneficial effect in the treatment of ophthalmic diseases.

It is a further object of the present invention to provide improved means for the prophylactic treatment of the ophthalmic system to improve function and to prevent or arrest diseases of the ophthalmic system.

It is another object to provide an electromagnetic treatment method of the above type having a broad-band, high spectral density electromagnetic field.

It is a further object of the present invention to provide a method of the above type in which amplitude modulation of the pulse burst envelope of the electromagnetic signal will induce coupling with a maximum number of relevant EMF-sensitive pathways in cells or tissues.

It is another object of the present invention to provide an improved method of enhancing soft tissue and hard tissue repair.

It is another object of the present invention to provide an improved method of increasing blood flow to affected tissues by modulating angiogenesis.

It is another object of the present invention to provide an improved method of increasing blood flow to enhance the viability and growth or differentiation of implanted cells, tissues and organs.

It is another object of the present invention to provide an improved method of increasing blood flow in cardiovascular diseases by modulating angiogenesis.

It is another object of the present invention to provide beneficial physiological effects through improvement of micro-vascular blood perfusion and reduced transudation.

It is another object of the present invention to provide an improved method of treatment of maladies of the bone and other hard tissue.

It is a still further object of the present invention to provide an improved means of the treatment of edema and swelling of soft tissue.

It is a still further object of the present invention to provide an improved means to enhance second messenger release.

It is another object of the present invention to provide a means of repair of damaged soft tissue.

It is yet another object of the present invention to provide a means of increasing blood flow to damaged tissue by modulation of vasodilation and stimulating neovascularization.

It is a yet further object of the present invention to provide an apparatus that can operate at reduced power levels as compared to those of related methods known in electromedicine and respective biofield technologies, with attendant benefits of safety, economics, portability, and reduced electromagnetic interference.

The above and yet other objects and advantages of the present invention will become apparent from the hereinafter set forth Brief Description of the Drawings and Detailed Description of the Invention.

### BRIEF DESCRIPTION OF THE DRAWINGS

Preferred embodiments of the present invention will be described below in more detail, with reference to the accompanying drawings:

FIG. 1 is a flow diagram of a electromagnetic treatment method for treatment of the ophthalmic tissue area according to an embodiment of the present invention;

FIG. 2 is a view of an electromagnetic treatment apparatus for ophthalmic tissue treatment according to a preferred embodiment of the present invention;

FIG. 3 is a block diagram of miniaturized circuitry according to a preferred embodiment of the present invention;

FIG. 4 is a block diagram of miniaturized circuitry according to another embodiment of the present invention;

FIG. 5 depicts a waveform delivered to eye target pathway structure according to a preferred embodiment of the present invention;

FIG. 6 is a bar graph illustrating PMF pre-treatment results;

FIG. 7 is a bar graph illustrating specific PMF signal results; and

FIG. 8 is a bar graph illustrating chronic PMF results.

### DETAILED DESCRIPTION OF THE INVENTION

Induced time-varying currents from PEMF or PRF devices flow in a target pathway structure such as a molecule, cell, tissue, and organ, and it is these currents that are a stimulus to which cells and tissues can react in a physiologically meaningful manner. The electrical properties of a target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and ion binding time constants of binding and other voltage sensitive membrane processes such as membrane transport. Knowledge of ion binding time constants allows SNR to be evaluated for any EMF signal configuration. A preferred embodiment according to the present invention uses ion binding time constants in the range of about 1 to about 100 msec.

Equivalent electrical circuit models representing various membrane and charged interface configurations have been derived. For example, in Calcium (“Ca2+”) binding, the change in concentration of bound Ca²⁺ at a binding site due to induced E may be described in a frequency domain by an impedance expression such as:

$${Z}_{b} ( ω ) = {R}_{ion} + \frac{1}{ⅈω {C}_{ion}}$$

which has the form of a series resistance-capacitance electrical equivalent circuit. Where ω is angular frequency defined as 2nf, where f is frequency, i=−1½, Zb(ω) is the binding impedance, and Rᵢₒₙ and Cᵢₒₙ are equivalent binding resistance and capacitance of an ion binding pathway. The value of the equivalent binding time constant, τᵢₒₙ=RᵢₒₙCᵢₒₙ, is related to a ion binding rate constant, k_{b}, via τᵢₒₙ=RᵢₒₙCᵢₒₙ=1/k_{b}. Thus, the characteristic time constant of this pathway is determined by ion binding kinetics.

Induced E from a PEMF or PRF signal can cause current to flow into an ion binding pathway and affect the number of Ca²⁺ ions bound per unit time. An electrical equivalent of this is a change in voltage across the equivalent binding capacitance Cᵢₒₙ, which is a direct measure of the change in electrical charge stored by Cion. Electrical charge is directly proportional to a surface concentration of Ca²⁺ ions in the binding site, that is storage of charge is equivalent to storage of ions or other charged species on cell surfaces and junctions. Electrical impedance measurements, as well as direct kinetic analyses of binding rate constants, provide values for time constants necessary for configuration of a PMF waveform to match a bandpass of target pathway structures. This allows for a required range of frequencies for any given induced E waveform for optimal coupling to target impedance, such as bandpass.

Ion binding to regulatory molecules is a frequent EMF target, for example Ca²⁺ binding to calmodulin (“CaM”). Use of this pathway is based upon acceleration of tissue repair, for example bone repair, wound repair, hair repair, and repair of other molecules, cells, tissues, and organs that involves modulation of growth factors released in various stages of repair. Growth factors such as platelet derived growth factor (“PDGF”), fibroblast growth factor (“FGF”), and epidermal growth factor (“EGF”) are all involved at an appropriate stage of healing. Angiogenesis and neovascularization are also integral to tissue growth and repair and can be modulated by PMF. All of these factors are Ca/CaM-dependent.

Utilizing a Ca/CaM pathway a waveform can be configured for which induced power is sufficiently above background thermal noise power. Under correct physiological conditions, this waveform can have a physiologically significant bioeffect.

Application of a Power SNR model to Ca/CaM requires knowledge of electrical equivalents of Ca2+ binding kinetics at CaM. Within first order binding kinetics, changes in concentration of bound Ca²⁺ at CaM binding sites over time may be characterized in a frequency domain by an equivalent binding time constant, τᵢₒₙ=RᵢₒₙCᵢₒₙ, where Rᵢₒₙ and Cᵢₒₙ are equivalent binding resistance and capacitance of the ion binding pathway. τᵢₒₙ is related to a ion binding rate constant, k_{b}, via τᵢₒₙ=RᵢₒₙCᵢₒₙ=1/k_{b}. Published values for k_{b} can then be employed in a cell array model to evaluate SNR by comparing voltage induced by a PRF signal to thermal fluctuations in voltage at a CaM binding site. Employing numerical values for PMF response, such as Vmax=6.5×10−7 sec⁻¹, [Ca²⁺]=2.5 μM, KD=30 μM, [Ca²⁺CaM]=KD([Ca²⁺]+[CaM]), yields k_{b}=665 sec⁻¹ (τᵢₒₙ=1.5 msec). Such a value for τᵢₒₙ can be employed in an electrical equivalent circuit for ion binding while power SNR analysis can be performed for any waveform structure.

According to an embodiment of the present invention a mathematical model can be configured to assimilate that thermal noise is present in all voltage dependent processes and represents a minimum threshold requirement to establish adequate SNR. Power spectral density, Sn(ω), of thermal noise can be expressed as:
Sₙ(ω)=4kTRe[Z_{M}(x,ω)]
where Z_{M}(x,ω) is electrical impedance of a target pathway structure, x is a dimension of a target pathway structure and Re denotes a real part of impedance of a target pathway structure. Z_{M}(x, ω) can be expressed as:

$${Z}_{M} ( x , ω ) = [ \frac{{R}_{e} + {R}_{i} + {R}_{g}}{γ} ] tanh ( γ x )$$

This equation clearly shows that electrical impedance of the target pathway structure, and contributions from extracellular fluid resistance (“Re”), intracellular fluid resistance (“Ri”) and intermembrane resistance (“Rg”) which are electrically connected to target pathway structures, all contribute to noise filtering.

A typical approach to evaluation of SNR uses a single value of a root mean square (RMS) noise voltage. This is calculated by taking a square root of an integration of Sₙ(ω)=4kTRe[Z_{M}(x,ω)] over all frequencies relevant to either complete membrane response, or to bandwidth of a target pathway structure. SNR can be expressed by a ratio:

$$S N R = \frac{\vert {V}_{M} ( ω ) \vert}{R M S}$$

where |V_{M}((ω)| is maximum amplitude of voltage at each frequency as delivered by a chosen waveform to the target pathway structure.

An embodiment according to the present invention comprises a pulse burst envelope having a high spectral density, so that the effect of therapy upon the relevant dielectric pathways, such as, cellular membrane receptors, ion binding to cellular enzymes and general transmembrane potential changes, is enhanced. Accordingly by increasing a number of frequency components transmitted to relevant cellular pathways, a large range of biophysical phenomena, such as modulating growth factor and cytokine release and ion binding at regulatory molecules, applicable to known tissue growth mechanisms is accessible. According to an embodiment of the present invention applying a random, or other high spectral density envelope, to a pulse burst envelope of mono-polar or bi-polar rectangular or sinusoidal pulses inducing peak electric fields between about 10⁻⁸ and about 100 V/cm, produces a greater effect on biological healing processes applicable to both soft and hard tissues.

According to yet another embodiment of the present invention by applying a high spectral density voltage envelope as a modulating or pulse-burst defining parameter, power requirements for such amplitude modulated pulse bursts can be significantly lower than that of an unmodulated pulse burst containing pulses within a similar frequency range. This is due to a substantial reduction in duty cycle within repetitive burst trains brought about by imposition of an irregular amplitude and preferably a random amplitude onto what would otherwise be a substantially uniform pulse burst envelope. Accordingly, the dual advantages, of enhanced transmitted dosimetry to the relevant dielectric pathways and of decreased power requirement are achieved.

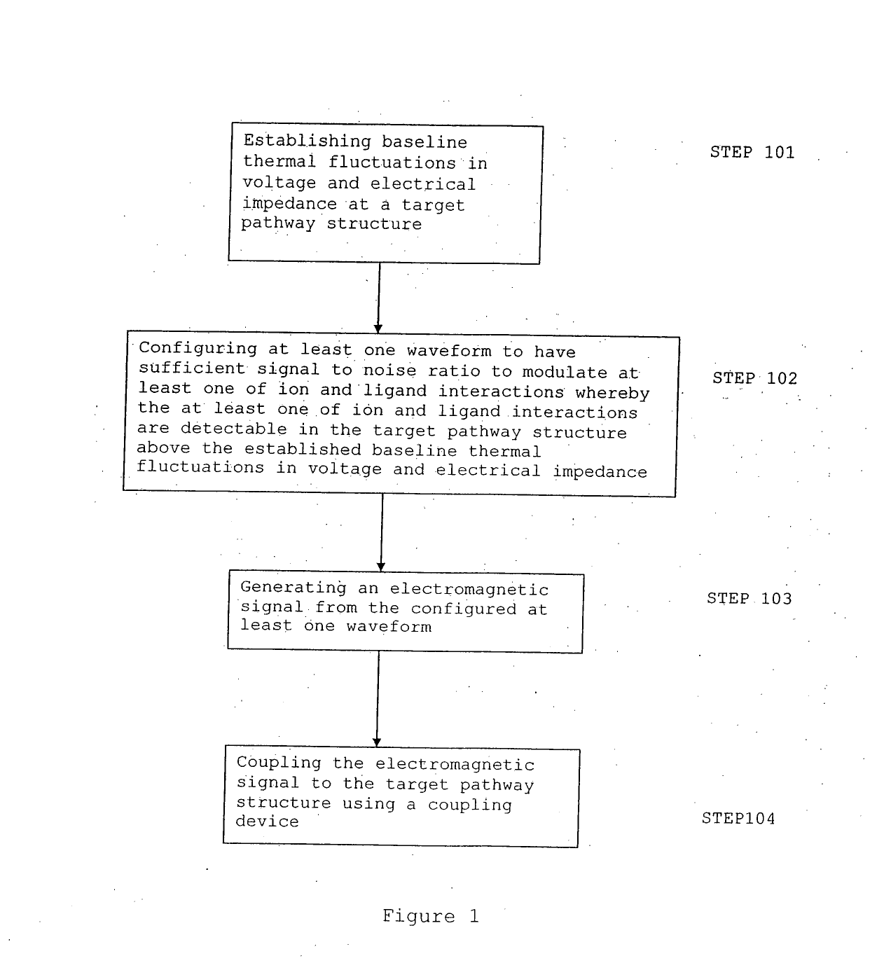

Referring to FIG. 1 wherein FIG. 1 is a flow diagram of a method for generating electromagnetic signals to be coupled to an eye according to an embodiment of the present invention, a target pathway structure such as ions and ligands, is identified. Establishing a baseline background activity such as baseline thermal fluctuations in voltage and electrical impedance, at the target pathway structure by determining a state of at least one of a cell and a tissue at the target pathway structure, wherein the state is at least one of resting, growing, replacing, and responding to injury. (STEP 101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance. (STEP 102) Generating an electromagnetic signal from the configured at least one waveform. (STEP 103) The electromagnetic signal can be generated by using at least one waveform configured by applying a mathematical model such as an equation, formula, or function having at least one waveform parameter that satisfies an SNR or Power SNR mathematical model such that ion and ligand interactions are modulated and the at least one configured waveform is detectable at the target pathway structure above its established background activity. Coupling the electromagnetic signal to the target pathway structure using a coupling device. (STEP 104) The generated electromagnetic signals can be coupled for therapeutic and prophylactic purposes. Since ophthalmic tissue is very delicate, application of electromagnetic signals using an embodiment according to the present invention is extremely safe and efficient since the application of electromagnetic signals is non-invasive.

A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a target pathway structure such as ions and ligands by output of a coupling device such as an electrode or an inductor, placed in close proximity to the target pathway structure using a positioning device. The coupling enhances modulation of binding of ions and ligands to regulatory molecules tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca²⁺ binding.

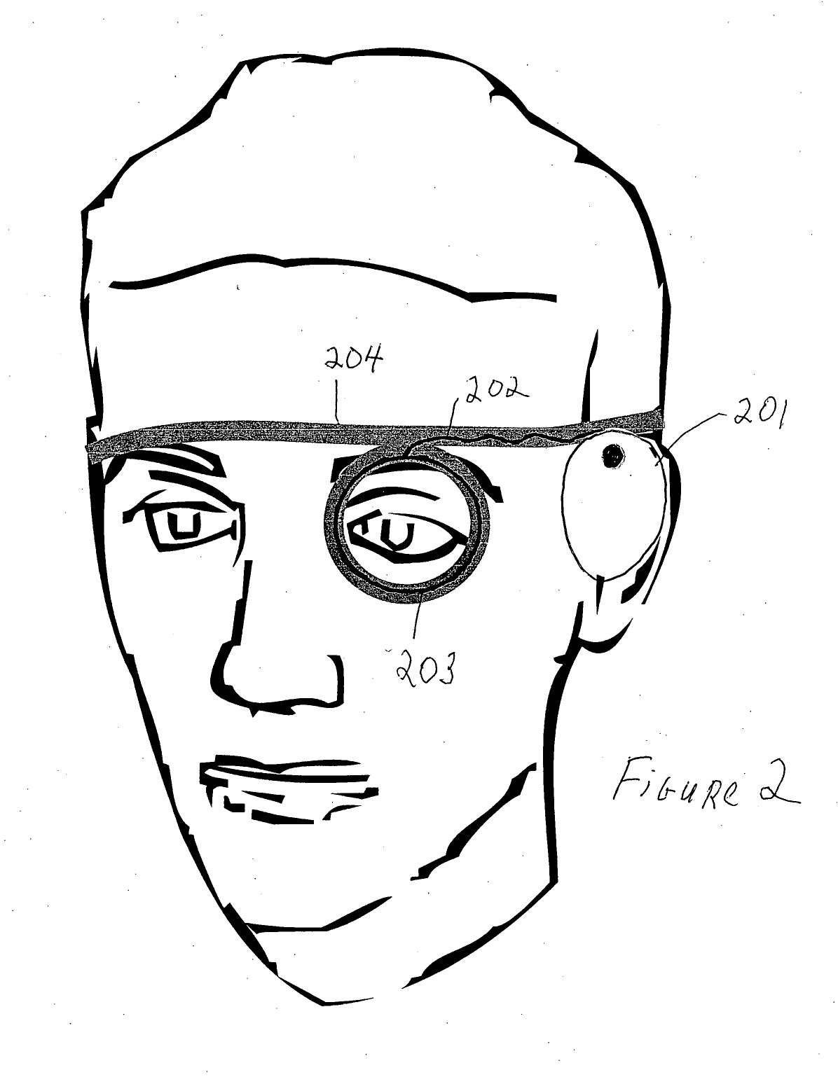

FIG. 2 illustrates a preferred embodiment of an apparatus according to the present invention. The apparatus is self-contained, lightweight, and portable. A miniature control circuit 201 is coupled to an end of at least one connector 202 such as wire however the control circuit can also operate wirelessly. The opposite end of the at least one connector is coupled to a generating device such as an electrical coil 203. The miniature control circuit 201 is constructed in a manner that applies a mathematical model that is used to configure waveforms. The configured waveforms have to satisfy a Power SNR model so that for a given and known target pathway structure, it is possible to choose waveform parameters that satisfy Power SNR so that a waveform is detectable in the target pathway structure above its background activity. A preferred embodiment according to the present invention applies a mathematical model to induce a time-varying magnetic field and a time-varying electric field in a target pathway structure such as ions and ligands, comprising about 0.001 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. A waveform configured using a preferred embodiment according to the present invention may be applied to a target pathway structure such as ions and ligands for a preferred total exposure time of under 1 minute to 240 minutes daily. However other exposure times can be used. Waveforms configured by the miniature control circuit 201 are directed to a generating device 203 such as electrical coils via connector 202. The generating device 203 delivers a pulsing magnetic field configured according to a mathematical model that can be used to provide treatment to a target pathway structure such as eye tissue. The miniature control circuit applies a pulsing magnetic field for a prescribed time and can automatically repeat applying the pulsing magnetic field for as many applications as are needed in a given time period, for example 10 times a day. The miniature control circuit can be configured to be programmable applying pulsing magnetic fields for any time repetition sequence. A preferred embodiment according to the present invention can be positioned to treat ophthalmic tissue by being incorporated with a positioning device 204 such as an eye-patch, eyeglasses, goggles, and monocles thereby making the unit self-contained. Coupling a pulsing magnetic field to a target pathway structure such as ions and ligands, therapeutically and prophylactically reduces inflammation thereby reducing pain and promotes healing in treatment areas. When electrical coils are used as the generating device 203, the electrical coils can be powered with a time varying magnetic field that induces a time varying electric field in a target pathway structure according to Faraday's law. An electromagnetic signal generated by the generating device 203 can also be applied using electrochemical coupling, wherein electrodes are in direct contact with skin or another outer electrically conductive boundary of a target pathway structure. Yet in another embodiment according to the present invention, the electromagnetic signal generated by the generating device 203 can also be applied using electrostatic coupling wherein an air gap exists between a generating device 203 such as an electrode and a target pathway structure such as ions and ligands. An advantage of the preferred embodiment according to the present invention is that its ultra lightweight coils and miniaturized circuitry allow for use with common physical therapy treatment modalities and at any location for which tissue growth, pain relief, and tissue and organ healing is desired. An advantageous result of application of the preferred embodiment according to the present invention is that tissue growth, repair, and maintenance can be accomplished and enhanced anywhere and at anytime. Yet another advantageous result of application of the preferred embodiment is that growth, repair, and maintenance of molecules, cells, tissues, and organs can be accomplished and enhanced anywhere and at anytime. A preferred embodiment according to the present invention delivers PEMF for application to ophthalmic tissue that is infected with diseases as macular degeneration, glaucoma, retinosa pigmentosa, repair and regeneration of optic nerve prophylaxis, and other related diseases.

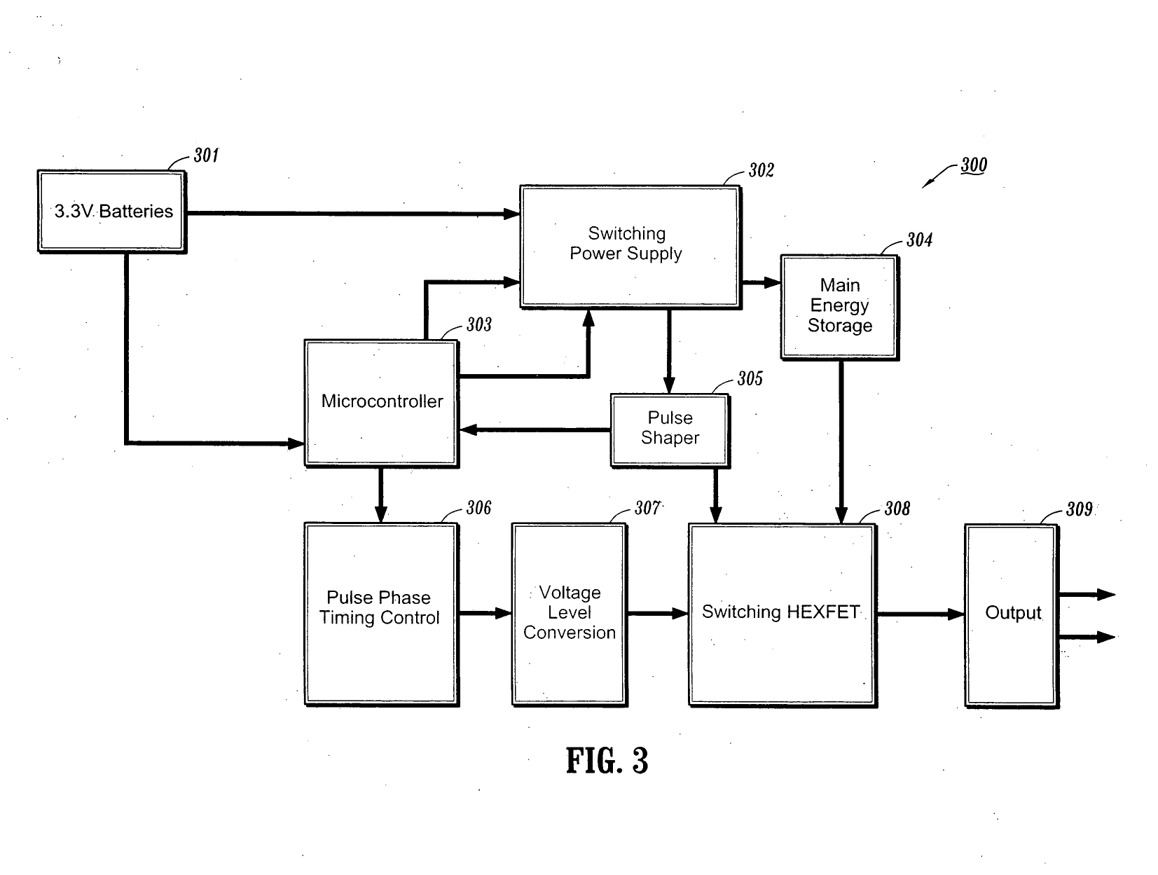

FIG. 3 depicts a block diagram of a preferred embodiment according to the present invention of a miniature control circuit 300. The miniature control circuit 300 produces waveforms that drive a generating device such as wire coils described above in FIG. 2. The miniature control circuit can be activated by any activation means such as an on/off switch. The miniature control circuit 300 has a power source such as a lithium battery 301. A preferred embodiment of the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. A preferred embodiment of the micro-controller 303 uses an 8 bit 4 MHz micro-controller 303 but other bit MHz combination micro-controllers may be used. The switching power supply 302 also delivers current to storage capacitors 304. A preferred embodiment of the present invention uses storage capacitors having a 220 uF output but other outputs can be used. The storage capacitors 304 allow high frequency pulses to be delivered to a coupling device such as inductors (Not Shown). The micro-controller 303 also controls a pulse shaper 305 and a pulse phase timing control 306. The pulse shaper 305 and pulse phase timing control 306 determine pulse shape, burst width, burst envelope shape, and burst repetition rate. In a preferred embodiment according to the present invention the pulse shaper 305 and phase timing control 306 are configured such that the waveforms configured are detectable above background activity at a target pathway structure by satisfying at least one of a SNR and Power SNR mathematical model. An integral waveform generator, such as a sine wave or arbitrary number generator can also be incorporated to provide specific waveforms. A voltage level conversion sub-circuit 308 controls an induced field delivered to a target pathway structure. A switching Hexfet 308 allows pulses of randomized amplitude to be delivered to output 309 that routes a waveform to at least one coupling device such as an inductor. The micro-controller 303 can also control total exposure time of a single treatment of a target pathway structure such as a molecule, cell, tissue, and organ. The miniature control circuit 300 can be constructed to be programmable and apply a pulsing magnetic field for a prescribed time and to automatically repeat applying the pulsing magnetic field for as many applications as are needed in a given time period, for example 10 times a day. A preferred embodiment according to the present invention uses treatments times of about 10 minutes to about 30 minutes.

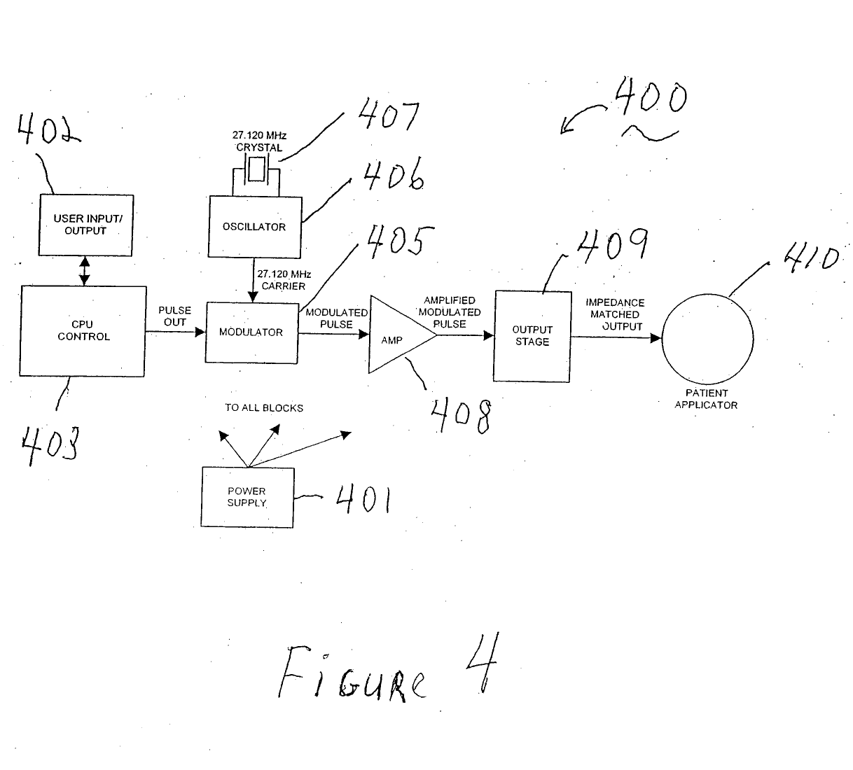

FIG. 4 depicts a block diagram of an embodiment according to the present invention of a miniature control circuit 400. The miniature control circuit 400 produces waveforms that drive a generating device such as wire coils described above in FIG. 2. The miniature control circuit can be activated by any activation means such as an on/off switch. The miniature control circuit 400 has a power source such as a lithium battery 401. In another embodiment according to the present invention the power source can be an external power source such as an electric current outlet such as an AC/DC outlet, coupled to the present invention for example by a plug and wire. A user input/output means 402 such as an on/off switch controls voltage to the miniature control circuit and is connected to a cpu-control 403. The cpu-control 403 creates a SNR EMF waveform by processing information provided to it via flash memory programmed having SNR EMF signal parameters such as pulse shape, burst width, burst envelope shape, and burst repetition rate. The waveform is pulse modulated by a modulator 405 interfacing with an oscillator 406 having a crystal 407 controlled by the cpu-control 403 according to the SNR EMF signal parameters programmed into the flash memory of the cpu-control 403. The oscillator 406 having a crystal 406 provides a carrier frequency. A preferred embodiment of the crystal is a 27.120 MHz crystal but other MHz crystals can be used. The modulated waveform is then amplified by an amp 408 and sent to an output stage means 409 where the amplified modulated waveform is matched to impedance via a R C circuit across a patient applicator 410 such as a coil. The patient applicator generates a SNR EMF signal to be delivered to a patient.

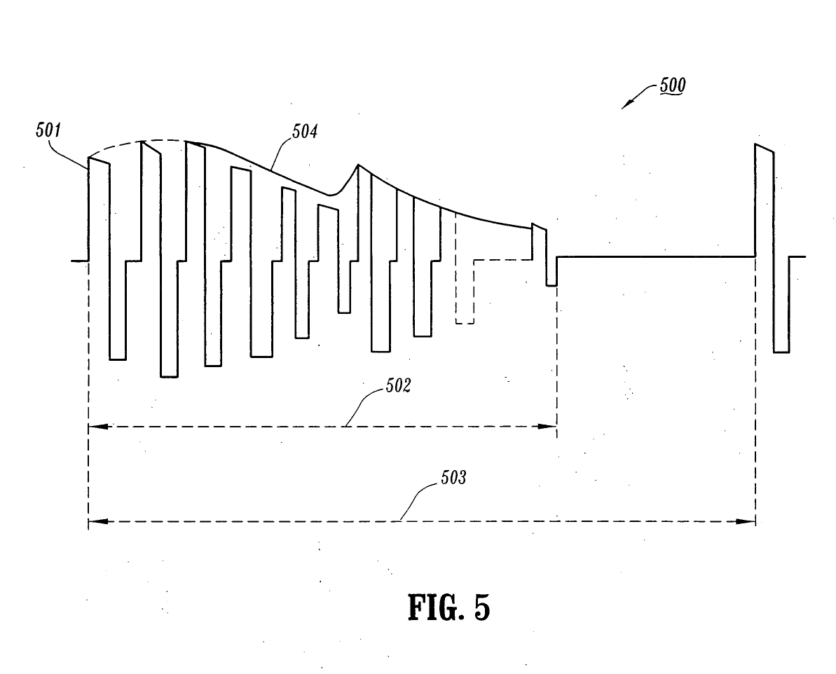

Referring to FIG. 5 an embodiment according to the present invention of a waveform 500 is illustrated. A pulse 501 is repeated within a burst 502 that has a finite duration 503. The duration 503 is such that a duty cycle which can be defined as a ratio of burst duration to signal period is between about 1 to about 10⁻⁵. A preferred embodiment according to the present invention utilizes pseudo rectangular 10 microsecond pulses for pulse 501 applied in a burst 502 for about 10 to about 50 msec having a modified 1/f amplitude envelope 504 and with a finite duration 503 corresponding to a burst period of between about 0.1 and about 10 seconds.

It is further intended that any other embodiments of the present invention that result from any changes in application or method of use or operation, method of manufacture, shape, size or material which are not specified within the detailed written description or illustrations and drawings contained herein, yet are considered apparent or obvious to one skilled in the art, are within the scope of the present invention.

### EXAMPLE 1

The Power SNR approach for PMF signal configuration has been tested experimentally on calcium dependent myosin phosphorylation in a standard enzyme assay. The cell-free reaction mixture was chosen for phosphorylation rate to be linear in time for several minutes, and for sub-saturation Ca²⁺ concentration. This opens the biological window for Ca²⁺/CaM to be EMF-sensitive. This system is not responsive to PMF at levels utilized in this study if Ca²⁺ is at saturation levels with respect to CaM, and reaction is not slowed to a minute time range. Experiments were performed using myosin light chain (“MLC”) and myosin light chain kinase (“MLCK”) isolated from turkey gizzard. A reaction mixture consisted of a basic solution containing 40 mM Hepes buffer, pH 7.0; 0.5 mM magnesium acetate; 1 mg/ml bovine serum albumin, 0.1% (w/v) Tween 80; and 1 mM EGTA12. Free Ca²⁺ was varied in the 1-7 μM range. Once Ca²⁺ buffering was established, freshly prepared 70 nM CaM, 160 nM MLC and 2 nM MLCK were added to the basic solution to form a final reaction mixture. The low MLC/MLCK ratio allowed linear time behavior in the minute time range. This provided reproducible enzyme activities and minimized pipetting time errors.

The reaction mixture was freshly prepared daily for each series of experiments and was aliquoted in 100 μL portions into 1.5 ml Eppendorf tubes. All Eppendorf tubes containing reaction mixture were kept at 0° C. then transferred to a specially designed water bath maintained at 37±0.1° C. by constant perfusion of water prewarmed by passage through a Fisher Scientific model 900 heat exchanger. Temperature was monitored with a thermistor probe such as a Cole-Parmer model 8110-20, immersed in one Eppendorf tube during all experiments. Reaction was initiated with 2.5 μM 32P ATP, and was stopped with Laemmli Sample Buffer solution containing 30 μM EDTA. A minimum of five blank samples were counted in each experiment. Blanks comprised a total assay mixture minus one of the active components Ca²⁺, CaM, MLC or MLCK. Experiments for which blank counts were higher than 300 cpm were rejected. Phosphorylation was allowed to proceed for 5 min and was evaluated by counting 32P incorporated in MLC using a TM Analytic model 5303 Mark V liquid scintillation counter.

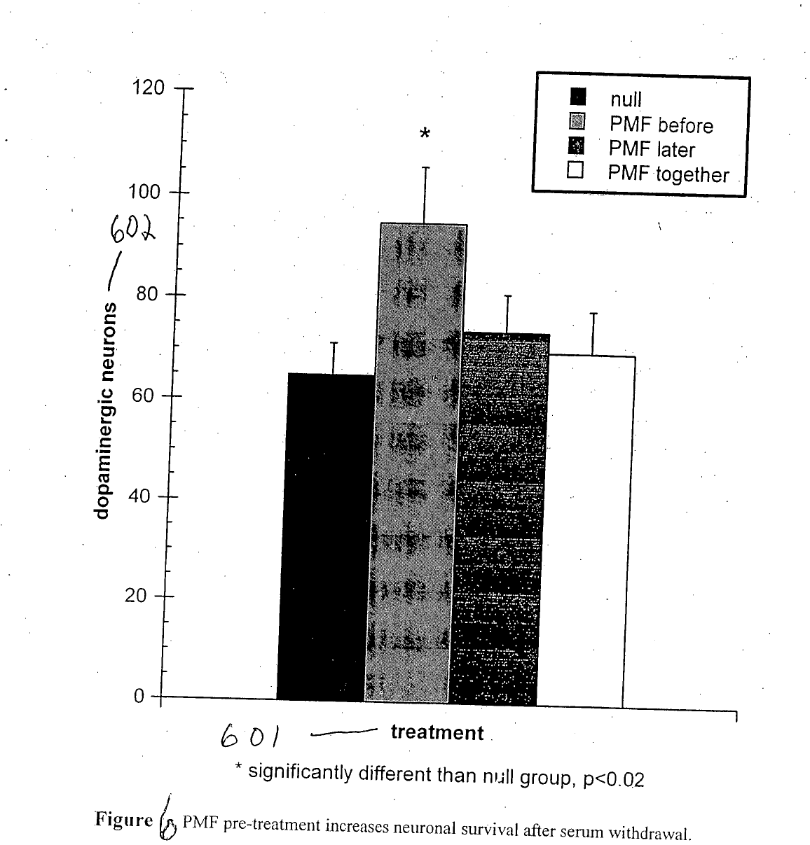

The signal comprised repetitive bursts of a high frequency waveform. Amplitude was maintained constant at 0.2 G and repetition rate was 1 burst/sec for all exposures. Burst duration varied from 65 μsec to 1000 μsec based upon projections of Power SNR analysis which showed that optimal Power SNR would be achieved as burst duration approached 500 μsec. The results are shown in FIG. 6 wherein burst width 601 in μsec is plotted on the x-axis and Myosin Phosphorylation 602 as treated/sham is plotted on the y-axis. It can be seen that the PMF effect on Ca²⁺ binding to CaM approaches its maximum at approximately 500 μsec, just as illustrated by the Power SNR model.

These results confirm that a PMF signal, configured according to an embodiment of the present invention, would maximally increase myosin phosphorylation for burst durations sufficient to achieve optimal Power SNR for a given magnetic field amplitude.

### EXAMPLE 2

According to an embodiment of the present invention use of a Power SNR model was further verified in an in vivo wound repair model. A rat wound model has been well characterized both biomechanically and biochemically, and was used in this study. Healthy, young adult male Sprague Dawley rats weighing more than 300 grams were utilized.

The animals were anesthetized with an intraperitoneal dose of Ketamine 75 mg/kg and Medetomidine 0.5 mg/kg. After adequate anesthesia had been achieved, the dorsum was shaved, prepped with a dilute betadine/alcohol solution, and draped using sterile technique. Using a #10 scalpel, an 8-cm linear incision was performed through the skin down to the fascia on the dorsum of each rat. The wound edges were bluntly dissected to break any remaining dermal fibers, leaving an open wound approximately 4 cm in diameter. Hemostasis was obtained with applied pressure to avoid any damage to the skin edges. The skin edges were then closed with a 4-0 Ethilon running suture. Post-operatively, the animals received Buprenorphine 0.1-0.5 mg/kg, intraperitoneal. They were placed in individual cages and received food and water ad libitum.

PMF exposure comprised two pulsed radio frequency waveforms. The first was a standard clinical PRF signal comprising a 65 μsec burst of 27.12 MHz sinusoidal waves at 1 Gauss amplitude and repeating at 600 bursts/sec. The second was a PRF signal reconfigured according to an embodiment of the present invention. For this signal burst duration was increased to 2000 μsec and the amplitude and repetition rate were reduced to 0.2 G and 5 bursts/sec respectively. PRF was applied for 30 minutes twice daily.

Tensile strength was performed immediately after wound excision. Two 1 cm width strips of skin were transected perpendicular to the scar from each sample and used to measure the tensile strength in kg/mm2. The strips were excised from the same area in each rat to assure consistency of measurement. The strips were then mounted on a tensiometer. The strips were loaded at 10 mm/min and the maximum force generated before the wound pulled apart was recorded. The final tensile strength for comparison was determined by taking the average of the maximum load in kilograms per mm2 of the two strips from the same wound.

The results showed average tensile strength for the 65 μsec 1 Gauss PRF signal was 19.3±4.3 kg/mm2 for the exposed group versus 13.0±3.5 kg/mm2 for the control group (p<0.01), which is a 48% increase. In contrast, the average tensile strength for the 2000 μsec 0.2 Gauss PRF signal, configured according to an embodiment of the present invention using a Power SNR model was 21.2±5.6 kg/mm2 for the treated group versus 13.7±4.1 kg/mm2 (p<0.01) for the control group, which is a 54% increase. The results for the two signals were not significantly different from each other.

These results demonstrate that an embodiment of the present invention allowed a new PRF signal to be configured that could be produced with significantly lower power. The PRF signal configured according to an embodiment of the present invention, accelerated wound repair in the rat model in a low power manner versus that for a clinical PRF signal which accelerated wound repair but required more than two orders of magnitude more power to produce.

### EXAMPLE 3

This example illustrates the effects of PRF electromagnetic fields chosen via the Power SNR method on neurons in culture.

Primary cultures were established from embryonic days 15-16 rodent mesencephalon. This area is dissected, dissociated into single cells by mechanical trituration, and cells are plated in either defined medium or medium with serum. Cells are typically treated after 6 days of culture, when neurons have matured and developed mechanisms that render them vulnerable to biologically relevant toxins. After treatment, conditioned media is collected.

Enzyme linked immunosorbent assays (“ELISAs”) for growth factors such as Fibroblast Growth Factor beta (“FGFb”) are used to quantify their release into the medium. Dopaminergic neurons are identified with an antibody to tyrosine hydroxylase (“TH”), an enzyme that converts the amino acid tyrosine to L-dopa, the precursor of dopamine, since dopaminergic neurons are the only cells that produce this enzyme in this system. Cells are quantified by counting TH+ cells in perpendicular strips across the culture dish under 100× magnification.

Serum contains nutrients and growth factors that support neuronal survival. Elimination of serum induces neuronal cell death. Culture media was changed and cells were exposed to PMF (power level 6, burst width 3000 μsec, and frequency 1 Hz). Four groups were utilized. Group 1 used No PMF exposure (null group). Group 2 used Pre-treatment (PMF treatment 2 hours before medium change). Group 3 used Post-treatment (PMF treatment 2 hours after medium change). Group 4 used Immediate treatment (PMF treatment simultaneous to medium change).

Results demonstrate a 46% increase in the numbers of surviving dopaminergic neurons after 2 days when cultures were exposed to PMF prior to serum withdrawal. Other treatment regimes had no significant effects on numbers of surviving neurons. The results are shown in FIG. 6 where type of treatment 601 is shown on the x-axis and number of neurons 602 is shown on the y-axis.

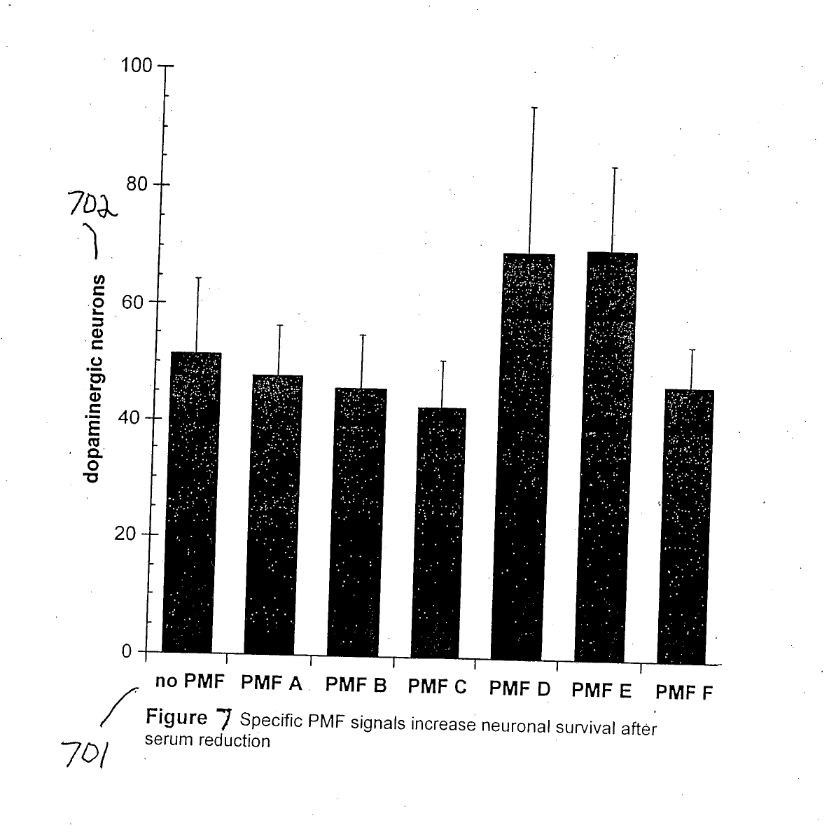

FIG. 7, where treatment 701 is shown on the x-axis and number of neurons 702 is shown on the y-axis, illustrates that PMF signals D and E increase numbers of dopaminergic neurons after reducing serum concentrations in the medium by 46% and 48% respectively. Both signals were configured with a burst width of 3000 μsec, and the repetition rates are 5/sec and 1/sec, respectively. Notably, signal D was administered in a chronic paradigm in this experiment, but signal E was administered only once: 2 hours prior to serum withdrawal, identical to experiment 1 (see above), producing effects of the same magnitude (46% vs. 48%). Since the reduction of serum in the medium reduces the availability of nutrients and growth factors, PMF induces the synthesis or release of these factors by the cultures themselves.

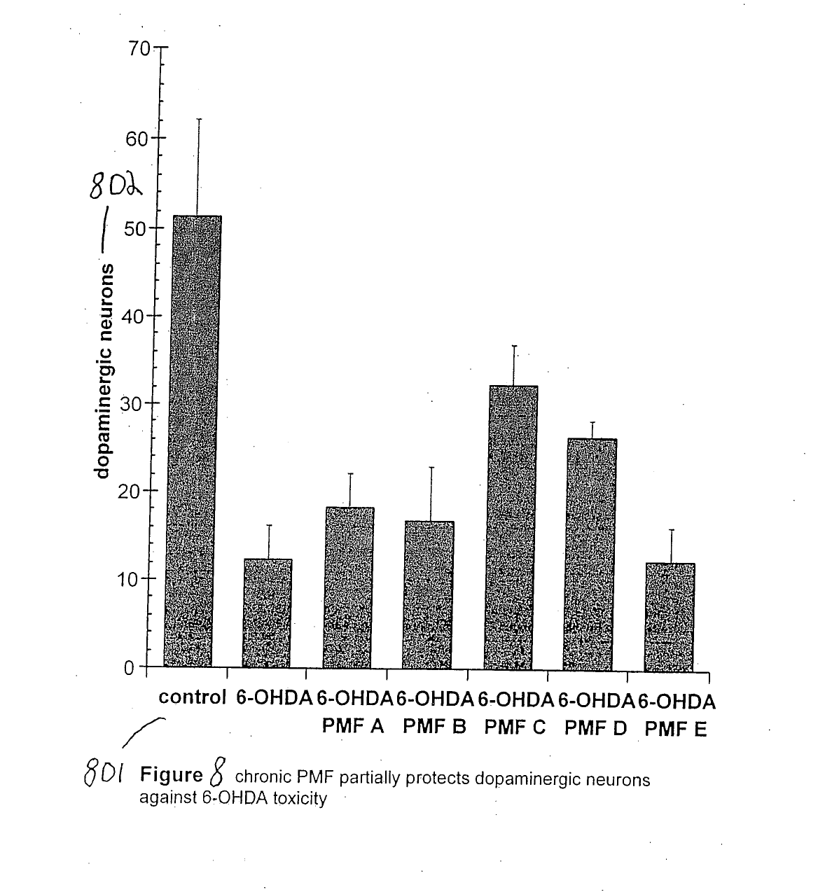

This portion of the experiment was performed to illustrate the effects of PMF toxicity induced by 6-OHDA, producing a well-characterized mechanism of dopaminergic cell death. This molecule enters cells via high affinity dopamine transporters and inhibits mitochondrial enzyme complex I, thus killing these neurons by oxidative stress. Cultures were treated with 25 μM 6-OHDA after chronic, or acute PMF exposure paradigms. FIG. 8 illustrates these results, where treatment 801 is shown on the x-axis and number of neurons 802 is shown on the y-axis. The toxin killed approximately 80% of the dopaminergic neurons in the absence of PMF treatment. One dose of PMF (power=6; burst width=3000 μsec; frequency=1/sec) significantly increased neuronal survival over 6-OHDA alone (2.6-fold; p≦0.02). This result has particular relevance to developing neuroprotection strategies for Parkinson's disease, because 6-OHDA is used to lesion dopaminergic neurons in the standard rodent model of Parkinson's disease, and the mechanism of toxicity is similar in some ways to the mechanism of neurodegeneration in Parkinson's disease itself.

### EXAMPLE 4

In this example electromagnetic field energy was used to stimulate neovascularization in an in vivo model. Two different signal were employed, one configured according to prior art and a second configured according to an embodiment of the present invention.

One hundred and eight Sprague-Dawley male rats weighing approximately 300 grams each, were equally divided into nine groups. All animals were anesthetized with a mixture of ketamine/acepromazine/Stadol at 0.1 cc/g. Using sterile surgical techniques, each animal had a 12 cm to 14 cm segment of tail artery harvested using microsurgical technique. The artery was flushed with 60 U/ml of heparinized saline to remove any blood or emboli.

These tail vessels, with an average diameter of 0.4 mm to 0.5 mm, were then sutured to the transected proximal and distal segments of the right femoral artery using two end-to-end anastomoses, creating a femoral arterial loop. The resulting loop was then placed in a subcutaneous pocket created over the animal's abdominal wall/groin musculature, and the groin incision was closed with 4-0 Ethilon. Each animal was then randomly placed into one of nine groups: groups 1 to 3 (controls), these rats received no electromagnetic field treatments and were killed at 4, 8, and 12 weeks; groups 4 to 6, 30 min. treatments twice a day using 0.1 gauss electromagnetic fields for 4, 8, and 12 weeks (animals were killed at 4, 8, and 12 weeks, respectively); and groups 7 to 9, 30 min. treatments twice a day using 2.0 gauss electromagnetic fields for 4, 8, and 12 weeks (animals were killed at 4, 8, and 12 weeks, respectively).

Pulsed electromagnetic energy was applied to the treated groups using a device constructed according to an embodiment of the present invention. Animals in the experimental groups were treated for 30 minutes twice a day at either 0.1 gauss or 2.0 gauss, using short pulses (2 msec to 20 msec) 27.12 MHz. Animals were positioned on top of the applicator head and confined to ensure that treatment was properly applied. The rats were reanesthetized with ketamine/acepromazine/Stadol intraperitoneally and 100 U/kg of heparin intravenously. Using the previous groin incision, the femoral artery was identified and checked for patency. The femoral/tail artery loop was then isolated proximally and distally from the anastomoses sites, and the vessel was clamped off. Animals were then killed. The loop was injected with saline followed by 0.5 cc to 1.0 cc of colored latex through a 25-gauge cannula and clamped. The overlying abdominal skin was carefully resected, and the arterial loop was exposed. Neovascularization was quantified by measuring the surface area covered by new blood-vessel formation delineated by the intraluminal latex. All results were analyzed using the SPSS statistical analysis package.

The most noticeable difference in neovascularization between treated versus untreated rats occurred at week 4. At that time, no new vessel formation was found among controls, however, each of the treated groups had similar statistically significant evidence of neovascularization at 0 cm2 versus 1.42±0.80 cm2 (p<0.001). These areas appeared as a latex blush segmentally distributed along the sides of the arterial loop. At 8 weeks, controls began to demonstrate neovascularization measured at 0.7±0.82 cm2. Both treated groups at 8 weeks again had approximately equal statistically significant (p<0.001) outcroppings of blood vessels of 3.57±1.82 cm2 for the 0.1 gauss group and of 3.77±1.82 cm2 for the 2.0 gauss group. At 12 weeks, animals in the control group displayed 1.75±0.95 cm2 of neovascularization, whereas the 0.1 gauss group demonstrated 5.95±3.25 cm2, and the 2.0 gauss group showed 6.20±3.95 cm2 of arborizing vessels. Again, both treated groups displayed comparable statistically significant findings (p<0.001) over controls.

These experimental findings demonstrate that electromagnetic field stimulation of an isolated arterial loop according to an embodiment of the present invention increases the amount of quantifiable neovascularization in an in vivo rat model. Increased angiogenesis was demonstrated in each of the treated groups at each of the sacrifice dates. No differences were found between the results of the two gauss levels tested as predicted by the teachings of the present invention.

Having described embodiments for an apparatus for applying electromagnetic signals to an eye and method for using same, it is noted that modifications and variations can be made by persons skilled in the art in light of the above teachings. It is therefore to be understood that changes may be made in the particular embodiments of the invention disclosed which are within the scope and spirit of the invention as defined by the appended claims.

What is claimed is:

1) An apparatus for altering the electromagnetic environment of ophthalmic tissues, cells, and molecules comprising:
an electromagnetic signal generating means for emitting signals comprising bursts of at least one of sinusoidal, rectangular, chaotic, and random waveforms, having a frequency content in a range of about 0.01 Hz to about 100 MHz at about 1 to about 100,000 waveforms per second, having a burst duration of 1 usec to 100 msec, and having a burst repetition rate from about 0.01 to about 1000 bursts/second, wherein the waveforms are adapted to have sufficient signal to noise ratio in respect of a given target pathway structure to modulate at least one of ion and ligand interactions in that target pathway structure, and
an electromagnetic signal coupling means for delivering the electromagnetic signal to the target pathway structure.

2) The apparatus of claim 1, wherein the signals comprise about 0.001 to about 100 msec bursts repeating at about 0.1 to about 10 pulses per second of about 1 to about 100 microsecond rectangular pulses.

3) The apparatus of claim 1, configured for providing an emitted signal having a peak signal amplitude at a target pathway structure in a range of about 1 μV/cm to about 100 mV/cm.

4) The apparatus of claim 1, wherein each signal burst envelope is a random function for providing a means to accommodate different electromagnetic characteristics of healing tissue.

5) The apparatus of claim 1, wherein the apparatus is configured for emitting a 20 millisecond pulse burst comprising about 0.1 microsecond to about 20 microsecond at least one of symmetrical and asymmetrical pulses repeating at about 1 to about 100 KHz within the burst.

6) The apparatus of claim 1, wherein the apparatus is configured for emitting an about 1 millisecond to an about 5 millisecond burst of 27.12 MHz sinusoidal waves repeating at about 1 to about 100 bursts/sec.

7) The apparatus of claim 1, wherein the coupling means is at least one of an inductive coupling means and a capacitive coupling means.

8) The apparatus of claim 1, wherein said target pathway structure includes at least one of an ophthalmic molecule, an ophthalmic cell, an ophthalmic tissue, and an ophthalmic organ.

9) An apparatus for altering the electromagnetic environment of ophthalmic tissues, cells, and molecules comprising:
A waveform configuration means for configuring at least one waveform to have sufficient signal to noise ratio to modulate at least one of ion and ligand interactions whereby the at least one of ion and ligand interactions are detectable in the target pathway structure above baseline thermal fluctuations in voltage and electrical impedance at a target pathway structure; and
A coupling device connected to the waveform configuration means by at least one connecting means for generating an electromagnetic signal from the configured at least one waveform and for coupling the electromagnetic signal to said target pathway structure whereby the at least one of ion and ligand interactions are modulated.

10) The apparatus of claim 9, wherein the at least one of ion and ligand interactions includes at least one of calcium ion binding and binding of calcium ions to calmodulin.

11) The apparatus of claim 8, wherein said target pathway structure includes at least one of an ophthalmic molecule, an ophthalmic cell, an ophthalmic tissue, and an ophthalmic organ.

12) The apparatus of claim 8, wherein the configuration means includes a configuration means for configuring at least one waveform having at least one of a frequency component parameter that configures said at least one waveform to be between about 0.01 Hz and about 100 MHz, a burst amplitude envelope parameter that follows an arbitrary amplitude function, a burst amplitude envelope parameter that follows a defined amplitude function, a burst width parameter that varies at each repetition according to an arbitrary width function, a burst width parameter that varies at each repetition according to a defined width function, a peak induced electric field parameter varying between about 1 μV/cm and about 100 mV/cm in said target pathway structure, and a peak induced magnetic electric field parameter varying between about 1 μT and about 0.1 T in said target pathway structure.

13) The apparatus of claim 12, wherein said defined amplitude function includes at least one of a 1/frequency function, a logarithmic function, a chaotic function and an exponential function.

14) The apparatus of claim 8, wherein the Signal to Noise Ratio includes a Power Signal to Noise Ratio.

15) The apparatus of claim 8, wherein said coupling device includes at least one of an inductive generating coupling device, a capacitive generating coupling device, an inductor, and an electrode.

16) The apparatus of claim 8, further comprising a positioning device to position the apparatus for delivering electromagnetic signals to at least one of an eye and an area in proximity to an eye.

17) The apparatus of claim 16, wherein said positioning device is at least one of an anatomical support, an anatomical wrap, and apparel.

18) The apparatus of claim 17, wherein said apparel includes at least one of garments, fashion accessories, eye-patches, eyeglasses, goggles, and monocles.

19) The apparatus of claim 8, wherein at least one of said waveform configuration means, connecting means, and coupling device is portable.

20) The apparatus of claim 8, wherein at least one of said waveform configuration means, connecting means, and coupling device is disposable.

21) The apparatus of claim 8, wherein at least one of said waveform configuration means, connecting means, and coupling device is implantable.

22) The apparatus of claim 8, wherein at least one of said waveform configuration means, connecting means, and coupling device is wireless.

23) A method for altering the electromagnetic environment of ophthalmic tissues, cells, and molecules comprising:
Establishing baseline thermal fluctuations in voltage and electrical impedance at a target pathway structure depending on a state of the ophthalmic tissue,
Configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance;
Generating an electromagnetic signal from the configured at least one waveform; and
Coupling the electromagnetic signal to the target pathway structure using a coupling device.

24) The method of claim 23, wherein the step of configuring at least one waveform to have sufficient signal to noise ratio to modulate at least one of ion and ligand interactions includes configuring at least one waveform to have sufficient signal to noise ratio to modulate calcium ion binding.

25) The method of claim 23, wherein the step of configuring at least one waveform to have sufficient signal to noise ratio to modulate at least one of ion and ligand interactions includes configuring at least one waveform to have sufficient signal to noise ratio to modulate binding of calcium ions to calmodulin.

26) The method of claim 23, wherein the step of configuring at least one waveform to have sufficient signal to noise ratio to modulate at least one of ion and ligand interactions includes configuring at least one waveform to have sufficient signal to noise ratio to match a bandpass of a second messenger at a target pathway structure whereby the second messenger modulates biochemical cascades related to tissue growth and repair.

27) The method of claim 23, wherein the step of establishing baseline thermal fluctuations in voltage and electrical impedance at a target pathway structure includes establishing baseline thermal fluctuations in voltage and electrical impedance at least one of an ophthalmic molecule, a ophthalmic cell, a ophthalmic tissue, and an ophthalmic organ.

28) The method of claim 23, wherein the step of coupling the electromagnetic signal to the target pathway structure using a coupling device includes coupling the electromagnetic signal to the target pathway structure using at least one of an inductive generating coupling device, a capacitive generating coupling device, an inductor, and an electrode.

29) The method of claim 23, wherein the step of coupling the electromagnetic signal to the target pathway structure includes coupling the electromagnetic signal to the target pathway structure to enhance the production of second messengers at the target pathway structure.

30) The method of claim 29, wherein the step of coupling the electromagnetic signal to the target pathway structure to enhance the production of second messengers at the target pathway structure includes coupling the electromagnetic signal to the target pathway structure to enhance the production of Nitric Oxide at the target pathway structure.

31) The method of claim 23, wherein the step of coupling the electromagnetic signal to the target pathway structure includes coupling the electromagnetic signal to the target pathway structure to enhance the production of growth factors at the target pathway structure.

32) The method of claim 23, wherein the step of coupling the electromagnetic signal to the target pathway structure includes coupling the electromagnetic signal to the target pathway structure to enhance the production of cytokines at the target pathway structure.

33) The method of claim 23, wherein the step of coupling the electromagnetic signal to the target pathway structure includes coupling the electromagnetic signal to the target pathway structure to enhance modulation of binding of at least one of ions and ligands to at least one of regulatory molecules, tissues, cells, and organs.

34) The method of claim 23, wherein the step of coupling the electromagnetic signal to the target pathway structure includes coupling the electromagnetic signal to the target pathway structure to provide treatment for at least one of macular degeneration, glaucoma, retinosa pigmentosa, optic nerve prophylaxis, and related ophthalmic diseases.
